(19) United States
(12) Patent Application Publication
(10) Pub. No.: US 2025/0302902 A1
(43) Pub. Date: Oct. 2, 2025

(54) System and Methods for Natural Products Extraction from Plant Biomass

(71) Applicant: Bioeutectics Corporation, Wilmington, DE (US)

(72) Inventors: Tomás Silicaro, Nordelta (AR); Sergio David Pasini Cabello, Ciudad Mendoza (AR); Maria Romina Canales, Junin mendoza (AR)

(73) Assignee: Bioeutectics Corporation, Wilmington, DE (US)

(21) Appl. No.: 19/093,744

(22) Filed: Mar. 28, 2025

Related U.S. Application Data
(60) Provisional application No. 63/571,490, filed on Mar. 29, 2024.

(51) Int. Cl. A61K 36/53; A61K 36/00; A61K 36/185; A61K 36/28; A61K 36/82; A61K 36/886; A61K 36/899; C11B 1/10

(52) U.S. Cl. CPC A61K 36/53; A61K 36/00; A61K 36/185; A61K 36/28; A61K 36/82; A61K 36/886; A61K 36/899; C11B 1/10; A61K 2236/333

(57) ABSTRACT

The present invention demonstrates the ability of NADES to extract natural products from plant biomass, for example, one can use corn husk, a common agricultural residue, as a promising lignocellulosic biomass resource for biofuel and biomaterial production. Corn husk is composed of three main components: lignin, cellulose, and hemicellulose with lignin acting as a natural shield, surrounding and protecting the cellulose fibers with its complex, water-insoluble structure. The NADES disclosed explores compositions that make access to the valuable cellulose a reality that can be used for further processing.

The currently disclosed process is superior because it has fewer environmental impact and toxicity issues due to the less harsh chemicals involved. The compositions and process demonstrate the potential of NADES as a more sustainable alternative to NaOH and Na₂S in the pulping process using NADES, which are formed by combining readily available and inexpensive chemicals, creating a solvent with unique properties.

The present application claims priority under 35 USC 119(e) to U.S. Provisional Application No. 63/571,490 filed Mar. 29, 2024, the contents of which are hereby incorporated by reference in its entirety.

### FIELD OF THE INVENTION

The present invention relates to exploring the potential of using various NADES (natural deep eutectic solvents) to extract various natural products from plant biomass, for example, using lignocellulosic biomass from a corn husk source and turning it into pulp, pectin extraction from various peels, oil from grapeseed, or protein or polyphenol extraction from plant biomass. The currently used methods of extraction employ harsh chemicals such as sodium hydroxide and sodium sulfide (known as Kraft pulping) so the present invention serves as a less harsh method of undergoing this chemical process.

### BACKGROUND OF THE INVENTION

Deep eutectic solvents or DESs are solutions of Lewis or Brønsted acids and bases which form a eutectic mixture. Deep eutectic solvents are highly tunable by varying the structure of the components or by varying the relative ratios of various components in the mixture. Because these are complicated systems that have widely varying properties, they have a wide variety of potential applications, including their use in catalysis, separation techniques, and electrochemical processes. The parent components of deep eutectic solvents tend to engage in complex hydrogen bonding networks, which means that the mixture tends to have significant freezing point depressions relative to the parent compounds/components in the mixture. Sometimes the individual components in the mixture may be solids at room temperature and atmospheric pressure, but when they are mixed together at room temperature and atmospheric pressure, the mixture may be a liquid that has a severely depressed freezing point (e.g., 10° C.).

The term “eutectic” was first coined in 1884 by British chemist and physicist Frederick Guthrie. The first generation of eutectic solvents were based on mixtures of quaternary ammonium salts with hydrogen bond donors such as amines and/or carboxylic acids. Natural deep eutectic solvents (NADES) are biologically based deep eutectic solvents which are composed of two or more compounds that are generally plant based primary metabolites, i.e., organic acids, sugars, alcohols, amines, and amino acids. Water may also be present as part of the solvent, as water is sometimes difficult to remove due to its inability to be easily evaporated.

Much of the study of eutectic solvents since Frederick Guthrie coined the term “eutectic” has involved solvent mixtures wherein at least one of the components is a metal-based solvent. However, the discharge of metals from these solvent systems has demonstrated many of the drawbacks associated with metal leaching, and its associated health, environmental, and safety related issues. Accordingly, there has been some recent interest in non-metal containing eutectic systems.

The plurality of uses for which eutectic solvents can be used is constantly developing. The below demonstrates more potential uses for these eutectic solvent systems.

### BRIEF SUMMARY OF THE INVENTION

The present invention relates to corn husk, a common agricultural residue, which is a promising lignocellulosic biomass resource for biofuel, paper production, and/or biomaterial production. It is composed of three main components: lignin, cellulose, and hemicellulose. Cellulose and hemicellulose are carbohydrate polymers and lignin is an aromatic-rich polymer that is insoluble in water and alcohol but weakly soluble in alkaline solutions. Lignin acts as a natural shield, surrounding and protecting the cellulosic fibers with its complex, water-insoluble structure. The properties of lignin makes it challenging to access the valuable cellulose for further processing.

The traditional pulping method is called “The Kraft pulping process” which currently reigns supreme in converting lignocellulosic biomass like corn husk into pulp. This method relies on a potent cocktail of sodium hydroxide (NaOH) and sodium sulfide (Na₂S) solutions. Under high temperatures and pressure, these chemicals break down the lignin, dismantling its protective barrier and liberating the cellulose fibers. While effective, the Kraft process raises concerns about environmental impact and toxicity due to the harsh chemicals involved. This invention explores the potential of using NADES as a more sustainable alternative to NaOH and Na₂S in the pulping process. NADES are formed by combining readily available and inexpensive chemicals, creating a solvent with unique properties.

Finally, the present invention relates to studying the properties of the biomass treated, like refrain curves and microscopic images.

### BRIEF DESCRIPTION OF THE SEVERAL VIEWS OF THE DRAWING

FIG. 1 depicts a photograph of corn husks prior to thermal treatment with any solvents.

FIG. 2 depicts a photograph of the lignocellulosic biomass which is attained after thermal treatment with a NADES solvent that comprises UGlH (a mix of urea (15-30%), glycerol (5-60%), and water (10-45%)).

FIG. 3 depicts a photograph of the lignocellulosic biomass which is attained after thermal treatment with a NADES solvent that comprises betaine and lactic acid in a 1 to 2 ratio.

FIG. 4 depicts a photograph of the lignocellulosic biomass which is attained after thermal treatment with a NADES solvent that comprises glycerol (15-50%), lactic acid (5-40%), oxalic acid (5-20%).

FIG. 5A depicts a FTIR spectrum of an apple pectin standard.

FIG. 5B depicts a FTIR spectrum of pectin extracted from orange peel.

FIG. 6A depicts a FTIR spectrum of crude grape seed oil.

FIG. 6B depicts a FTIR spectrum of commercial grape seed oil.

FIG. 6C depicts a FTIR spectrum of grape seed oil extracted with hexane.

FIG. 6D depicts a FTIR spectrum of grape seed oil extracted with UL.

FIG. 7A depicts test tube results of bio-extracts with LGH wherein in order from left to right are chamomile, green tea, rosemary, aloe vera, and thyme.

FIG. 7B depicts test tube results of bio-extracts with LGH wherein in order from left to right are yerba mate, horsetail, lemon balm, oatmeal, and nettle.

FIG. 7C depicts test tube results of bio-extracts with UGlH wherein in order from left to right are chamomile, green tea, rosemary, aloe vera, and thyme.

FIG. 7D depicts test tube results of bio-extracts with UGlH wherein in order from left to right are yerba mate, horsetail, lemon balm, oatmeal, and nettle.

FIG. 7E depicts test tube results of bio-extracts with UL wherein in order from left to right are chamomile, green tea, rosemary, aloe vera, and thyme.

FIG. 7F depicts test tube results of bio-extracts with UL wherein in order from left to right are yerba mate, horsetail, lemon balm, oatmeal, and nettle.

FIG. 7G depicts test tube results of bio-extracts with BeL wherein in order from left to right are chamomile, green tea, rosemary, aloe vera, and thyme.

FIG. 7H depicts test tube results of bio-extracts with BeL wherein in order from left to right are yerba mate, horsetail, lemon balm, oatmeal, and nettle.

FIG. 7I depicts test tube results of bio-extracts with ArgCH wherein in order from left to right are chamomile, green tea, rosemary, aloe vera, and thyme.

FIG. 7J depicts test tube results of bio-extracts with ArgCH wherein in order from left to right are yerba mate, horsetail, lemon balm, oatmeal, and nettle.

FIG. 7K depicts test tube results of bio-extracts with BeUH wherein in order from left to right are chamomile, green tea, rosemary, aloe vera, and thyme.

FIG. 7L depicts test tube results of bio-extracts with BeUH wherein in order from left to right are yerba mate, horsetail, lemon balm, oatmeal, and nettle.

FIG. 7M depicts test tube results of bio-extracts with BePGG wherein in order from left to right are chamomile, green tea, rosemary, aloe vera, and thyme.

FIG. 7N depicts test tube results of bio-extracts with BePGG wherein in order from left to right are yerba mate, horsetail, lemon balm, oatmeal, and nettle.

FIG. 7O depicts test tube results of bio-extracts with LSH wherein in order from left to right are chamomile, green tea, rosemary, aloe vera, and thyme.

FIG. 7P depicts test tube results of bio-extracts with LSH wherein in order from left to right are yerba mate, horsetail, lemon balm, oatmeal, and nettle.

FIG. 7Q depicts test tube results of bio-extracts with LGl wherein in order from left to right are chamomile, green tea, rosemary, aloe vera, and thyme.

FIG. 7R depicts test tube results of bio-extracts with LGl wherein in order from left to right are yerba mate, horsetail, lemon balm, oatmeal, and nettle.

FIG. 8 depicts a bar graph of a Matricaria chamomilla extraction profile.

FIG. 9A depicts a bar graph of a quercetin extraction profile from Aloe barbadensis.

FIG. 9B depicts a bar graph of a theophylline extraction profile from Aloe barbadensis.

FIG. 10 depicts a bar graph of an Avena sativa extraction profile.

FIG. 11 depicts a bar graph of a theophylline extraction profile from Avena sativa.

FIG. 12 depicts a bar graph of a Chamaemelum nobile extraction profile.

FIG. 13A depicts a bar graph of a caffeic acid extraction profile from Chamaemelum nobile.

FIG. 13B depicts a bar graph of a theophylline extraction profile from Chamaemelum nobile.

FIG. 13C depicts a bar graph of a gallic acid extraction profile from Chamaemelum nobile.

FIG. 13D depicts a bar graph of a cinnamic acid extraction profile from Chamaemelum nobile.

FIG. 14 depicts a bar graph of Camellia sinensis extraction profile.

FIG. 15A depicts a bar graph of theophylline extraction profile from Camellia sinensis.

FIG. 15B depicts a bar graph of chlorogenic acid extraction profile from Camellia sinensis.

FIG. 16 depicts a bar graph of Thymus vulgaris extraction profile.

FIG. 17A depicts a bar graph of caffeic acid extraction profile from Thymus vulgaris.

FIG. 17B depicts a bar graph of ferulic acid extraction profile from Thymus vulgaris.

FIG. 17C depicts a bar graph of a chlorogenic acid extraction profile from Thymus vulgaris.

FIG. 18 depicts a bar graph of an Urtica dioica extraction profile.

FIG. 19A depicts a bar graph of theophylline extraction profile from Urtica dioica.

FIG. 19B depicts a bar graph of a chlorogenic acid extraction profile from Urtica dioica.

FIG. 20 depicts a bar graph of a Melissa officinalis extraction profile.

FIG. 21 depicts a bar graph of caffeic acid extraction profile from Melissa officinalis.

FIG. 22 depicts a bar graph of a Rosmarinus officinalis extraction profile.

FIG. 23A depicts a bar graph of caffeic acid extraction profile from Rosmarinus officinalis.

FIG. 23B depicts a bar graph of gallic acid extraction profile from Rosmarinus officinalis.

FIG. 23C depicts a bar graph of ferulic acid extraction profile from Rosmarinus officinalis.

FIG. 24 depicts a bar graph of an Equisetum arvense extraction profile.

FIG. 25A depicts a bar graph of a caffeine extraction profile from Equisetum arvense.

FIG. 25B depicts a bar graph of a ferulic acid extraction profile from Equisetum arvense.

FIG. 25C depicts a bar graph of a chlorogenic acid extraction profile from Equisetum arvense.

FIG. 25D depicts a bar graph of a gallic acid extraction profile from Equisetum arvense.

### DETAILED DESCRIPTION OF THE INVENTION

### Laboratory Tests

Corn was used as a sample. The biomass was heated within an aqueous solution in different NADES concentrations. Mimicking the Kraft method.
Extraction of lignin from corn with several NADES

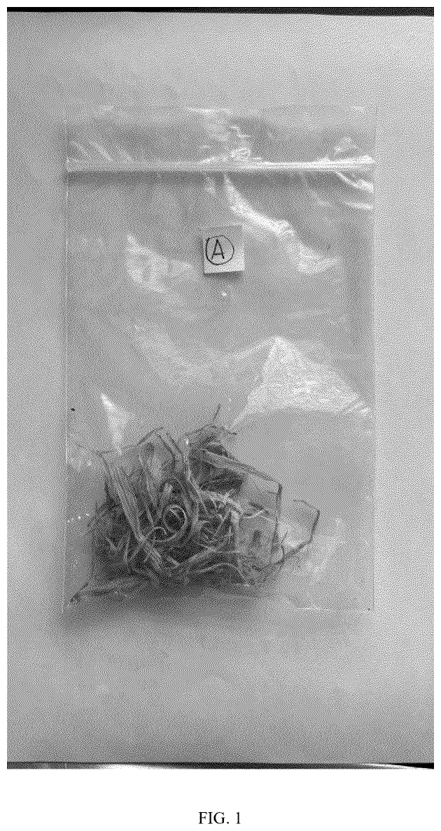

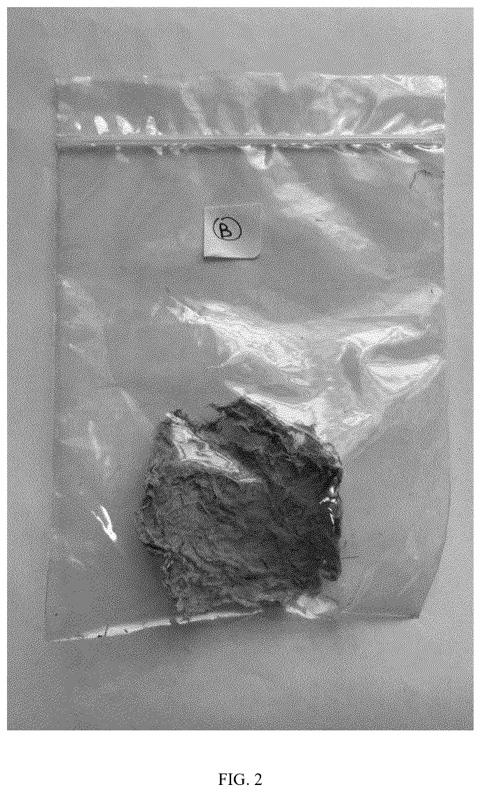

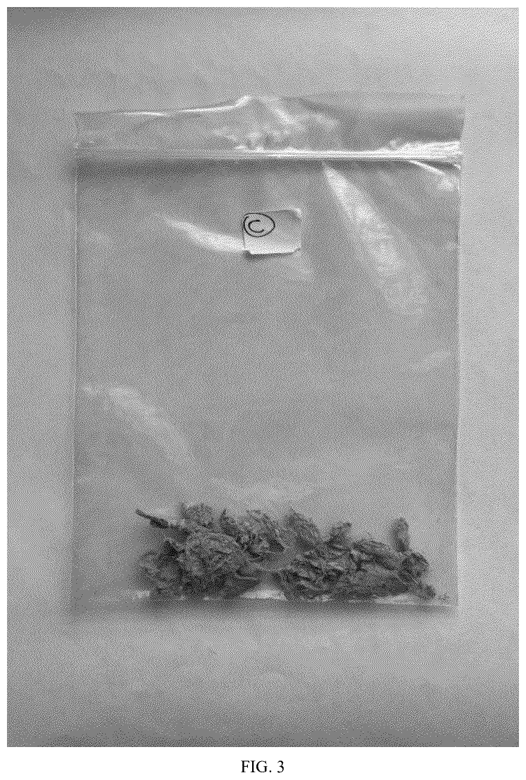

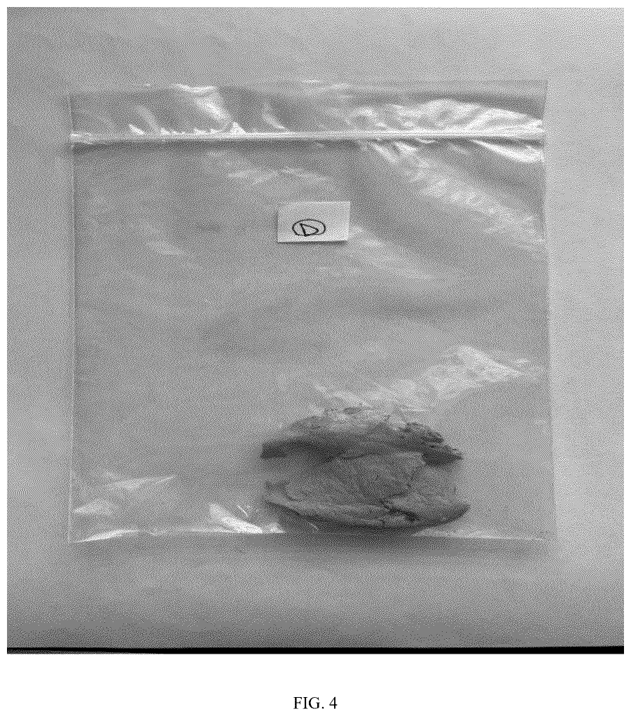

The following methodology was used to extract lignin from the biomass:
a. Sample/solvent mass ratio: 20% w/w
b. Temperature: 90° C.-120° C.
c. Extraction time: 2-4 hours
   Extraction with Several NADES
   Several NADES were tested as solvents for corn husk pretreatment. The composition of these solvents is as follows: FIG. 2 UGlH (a mix of urea (15-30%), glycerol (5-60%), and water (35-75%)).
   FIG. 3 BeL: betaine, lactic acid (1:2) and FIG. 4 GILOxA (a mix of glycerol (15-50%), lactic acid (5-40%), and oxalic acid (5-20%)).
   FIGS. 1-4 show the biomass without any treatment (FIG. 1) and after thermal treatment with the various solvents of the present invention as disclosed above (FIGS. 2-4).

Based on these experiments, UGlH and BeL showed a better effect on lignin extraction. Future experiments will evaluate the biomass pretreatment's physical properties to determine the process's efficacy.

Experiments were also conducted to ascertain the effectiveness of extracting pectin from various fruit products using different NADES.

Pectin Extraction from Fruit Peels

Currently, pectins are widely used in the cosmetic, pharmaceutical, and food industries, mainly as texturizing, emulsifying, stabilizing, and gelling agents. Pectins represent a group of structural heteropolysaccharides, composed mainly of covalently α-1,4-linked D-galacturonic acid (GalA) units, found in the primary cell wall of fruit products.

Currently, fruit peels can be considered as raw materials to produce value-added pectins. Conventional extraction methods consist of thermal acid hydrolysis with mineral acids such as nitric, sulfuric, and hydrochloric acids. During the heating process, pectin is extracted at pH 1.5-3 and at a temperature between 75 and 100° C. for 1-3 h. Considering the emerging approach of a sustainable process as disclosed in the present invention, the focus of the present invention involves shifting from mineral to organic acids. In this context, NADES can be used as an excellent replacement for mineral acids. The following experiments were conducted.

### Laboratory Tests

Orange peel (Citrus sinensis) was used as a sample. The biomass was heated within an aqueous solution using different NADES concentrations.

The following methodology was used to extract pectin from the biomass:
a. The sample to solvent mass ratio was 1-5% w/w.
b. The temperature used was between 70° C.-90° C.
c. The extraction time was between 1-3 hours.

After acid hydrolysis, pectin was purified by alcohol precipitation. Several NADES were tested as pectin extractors from orange peels. The composition of these solvents was as follows: LGH: lactic acid (5-70%), glucose (5-30%), water (5-10%); BeL: betaine (10-40%), lactic acid (60-90); MAL: Malic acid (10-30%), Lactic acid (70-90%); TAMAL: Tartaric acid (5-10%), Malic acid (10-30%), Lactic acid (70-90%) and GILOxA: Glycerol (15-50%), Lactic Acid (5-40%), Oxalic Acid (5-20%).

The following table 3 summarizes the yields for each solvent.

TABLE 3

NADES/% w/w

| | 1 | 3 |
|---|---|---|
| Conventional (Sulfuric Acid) | 11.2 | 12.3 |
| LGH | 8.2 | 9.4 |
| BeL | 5.3 | 9.0 |
| MAL | 4.9 | 12 |
| GILOxA | 7.9 | 11.2 |
| TAMAL/GILOxA | 10.2 | 12.3 |

Based on the above study, it was found that products/solvents can be used as an alternative to the caustic sulfuric acid/chloridric acid (hydrochloric acid) mix to give results in the pectin extraction with comparable yields.

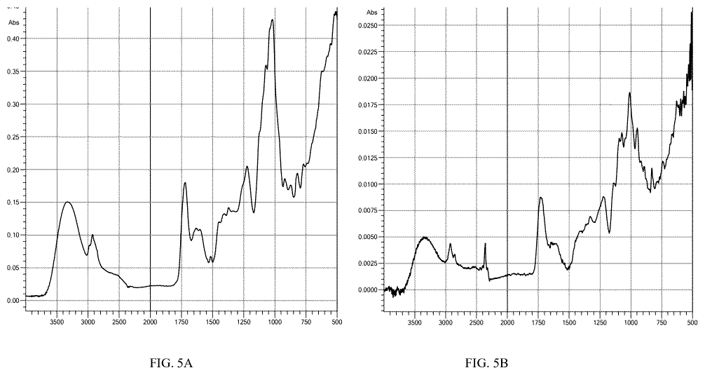

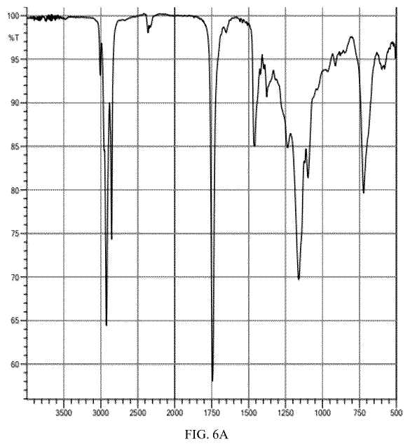

Additionally, FTIR-ATR (Fourier-transformed infrared spectroscopy (FTIR) with a Single Attenuated Total Reflectance accessory (QATR-S) IRSpirit, Shimadzu Corporation, Japan) was performed to confirm the presence of pectin after extraction. FIGS. 5A and 5B show the FTIR absorbance spectra for the sample and for a standard of apple pectin (Spectrum Chemical, NJ, USA). The results as shown in FIG. 5A and FIG. 5B demonstrate and indicate that pectin was extracted.

Extraction of Oil from Grape Seeds

The extraction of vegetable oils is often carried out using organic solvents. Generally, one can attain 99.5% to 99.3% of oil content recovered. The traditional methodology and process is generally applied to vegetable materials with low oil content, or cakes obtained from materials with high oil content but which have been previously pressed.

The use of organic solvents, in this case hexane, makes the extraction process unsafe, since it is a highly flammable substance and its use generally necessitates a subsequent refining process to eliminate other toxic components that are extracted.

The proposed process mediated by NADES is highly safe and does not impart the oil with toxic consumption properties. In addition to the fact that NADES imparts no toxic characteristics, the NADES used are advantageous in that they are safe, non-flammable, inexpensive, and also biodegradable.

### Laboratory Tests

Grape seed pellets were used as a sample, which was heated to 60° C. with NADES and then centrifuged to obtain the oil by entrainment. One characteristic that makes extraction by using NADES advantageous is that the NADES is insoluble in the oil.

The following methodology was used for grape seed oil extraction:
a. Sample/solvent ratio: 25% w/w.
b. Temperature: 60° C.
c. Extraction time: 30 minutes
   The best results were obtained with UL (urea 6-40% and lactic acid 60-94%). The yields obtained with the traditional method and the method used with NADES are shown in table 4 below:

TABLE 4

| Solvent | yield % w/w |
|---|---|
| Conventional (Hexane) | 11 |
| Press | 0.8 |
| UL | 6 |

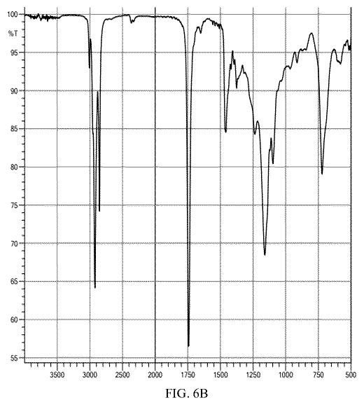

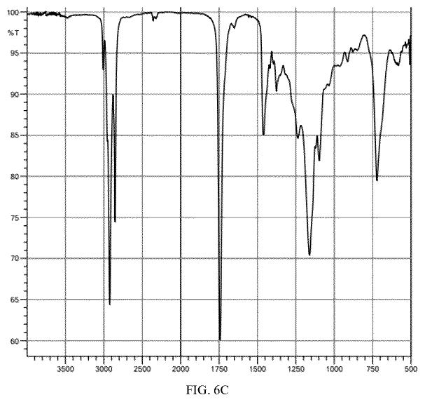

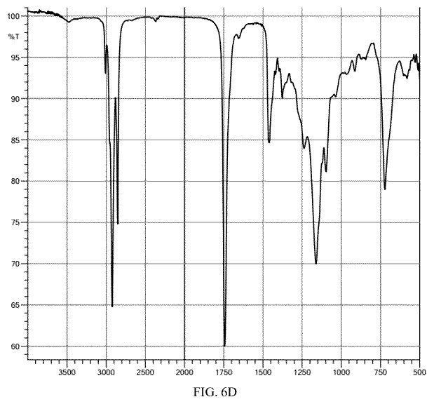

FIGS. 6A-6D show a comparison of the data obtained by ATR-IR (Fourier-transformed infrared spectroscopy (FTIR) with Single Attenuated Total Reflectance accessory (QATR-S) IRSpirit, Shimadzu Corporation, Japan), of the commercial grape seed oil, crude grape seed oil, oil extracted with hexane (FIG. 6C) and the oil extracted with UL (FIG. 6D):
Comparison of Crude Oil, Commercial Oil, Oil Extracted with Hexane in the Laboratory and Oil Extracted with UL

FIG. 6: A) crude oil B) commercial oil C) oil extracted with hexane and D) oil extracted with UL-01.

Additionally, a physicochemical characterization of the different oils enumerated above was carried out, which is detailed in Table 5 below:

TABLE 5

| | | Density |
|---|---|---|
| Sample | Brix | (g/ml) |
| Crude oil | 73.4 | 0.922 |
| Oil extracted with UL | 73.1 | 0.939 |
| Commercial oil | 73.6 | 0.913 |

The oil extracted with UL achieved favorable results demonstrating that extraction of the oil has occurred.

### Protein Extraction and Isolation

Protein extraction and precipitation are two methods used to obtain protein isolates from many plant sources. Traditional extraction is carried out with sodium hydroxide to give an alkaline medium and subsequently, an acid such as sulfuric acid is added to precipitate the proteins.

This process requires the handling of strong acids and bases, which makes it unsafe for the operator and these compounds tend to be present in the final product.

The proposed process mediated by NADES is a safe method, and the components used as pH regulators are food grade, which means that they have little to no negative effect on the finished product.

### Laboratory Tests

Non-fat soybean meal was used as a source of protein. For alkaline extraction, LysMdH: L-Lysine (10-30%), maltodextrin (2-15%), and water (40-80%) was used, which had the pH adjusted to 9. Subsequently, MAL: malic acid (20-60%) and lactic acid (40-80%) was used for precipitation, where the pH was adjusted to 4.5.

The following methodology was used for grape seed oil extraction:
a. Sample: 50 g
b. Alkaline solvent: 23 ml
c. Acid solvent: 2.5 ml
b. Temperature: 25° C.
c. Extraction time: 30 minutes
   Table 6 below shows the results obtained with the traditional method and the method using NADES.

TABLE 6

| Method | Traditional | NADES |
|---|---|---|
| Flour (g) | 50 | 50 |
| Alkaline solvent | 1 | 21 |
| consumption (mL) | | |
| Acid solvent | 3 | 2.5 |
| consumption (mL) | | |
| Yield % p/p | 18.3 | 18.4 |

The results as shown in Table 6 illustrate that by using NADES instead of the traditional methodology, one is able to attain comparable (if not slightly better) results.

### Use of NADES for the Extraction of Polyphenols

### Objectives

To determine and/or isolate polyphenolic compounds from medicinal plants using natural deep eutectic solvents.
To compare hydrophilic eutectic systems vs a hydro-glyceric mixture, which is currently utilized in the extract industry.

### Laboratory Tests

### NADES Preparation

All eutectic systems were prepared by stirring in a magnetic stirrer at 60° C. for 60 min, with continuous stirring until the formation of a transparent liquid was observed. The following eutectic systems were employed for the maceration extractions:
LGH: lactic acid, dextrose, and water (molar relation: 5:1:9)
UGlH: urea, glycerol, and water (molar relation: 1:1.2:1.2)
UL: urea and lactic acid (molar relation: 1:2)
LGl: lactic acid and glycerol (molar relation: 1:1)
BeL: betaine and lactic acid (molar relation: 1:1)
ArgCH: L-arginine, citric acid, and water (molar relation: 1:1:6.45)
ArgLH: L-arginine, lactic acid, and water (molar relation: 1:1:9)
BeGlG: betaine, glycerol, and dextrose (molar relation: 4:20:1)
BeGl: betaine, glycerol (molar relation: 1:4)
BePgH: betaine, polyethylene glycol and water (molar relation: 4:20:1)
LSH: lactic acid, sorbitol, and water (molar relation: 5:1:9)

### Maceration Extraction Procedure

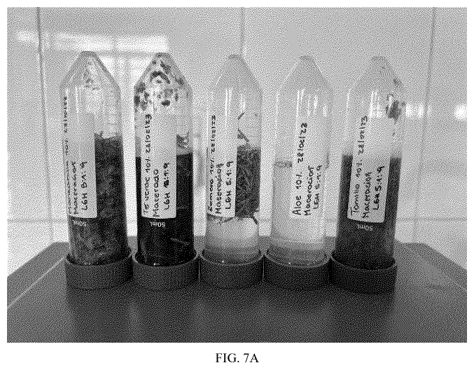

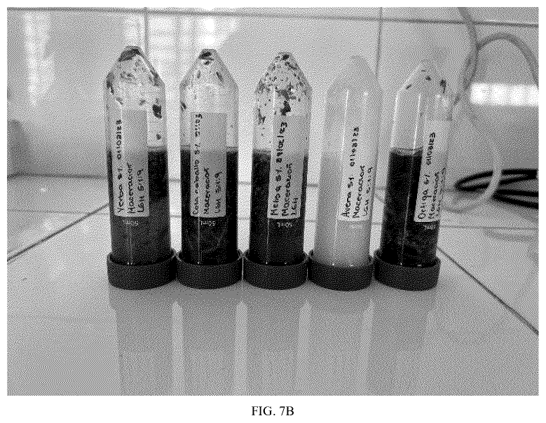

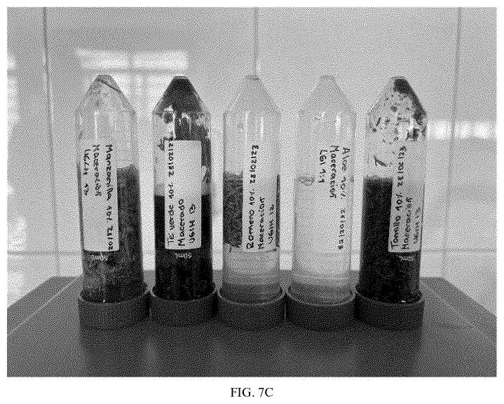

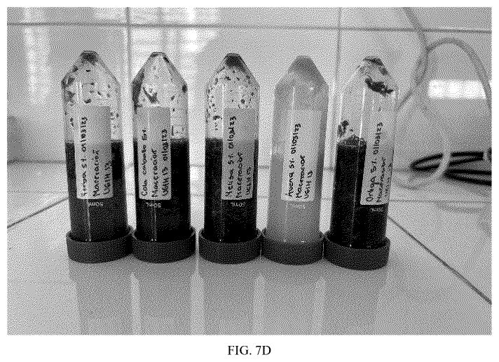

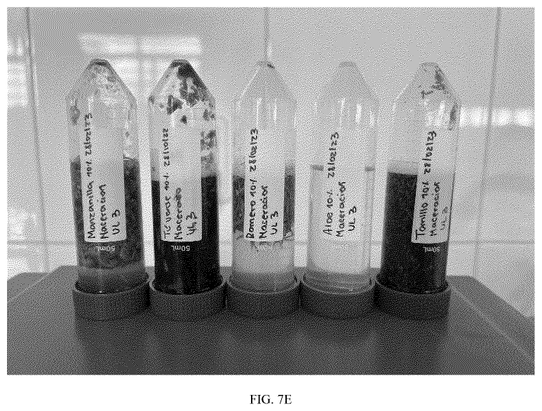

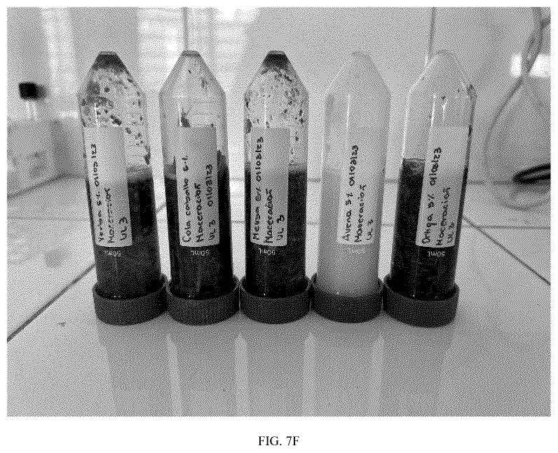

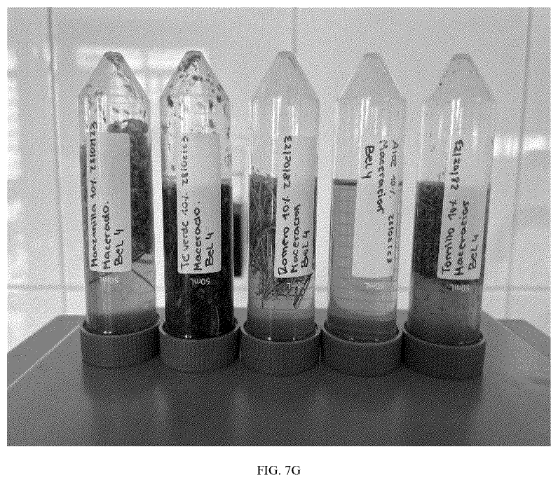

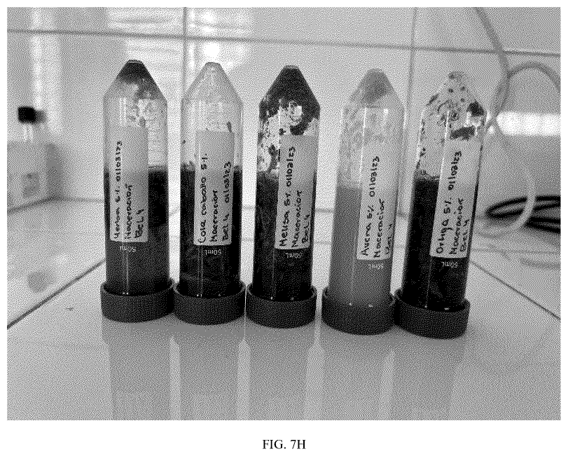

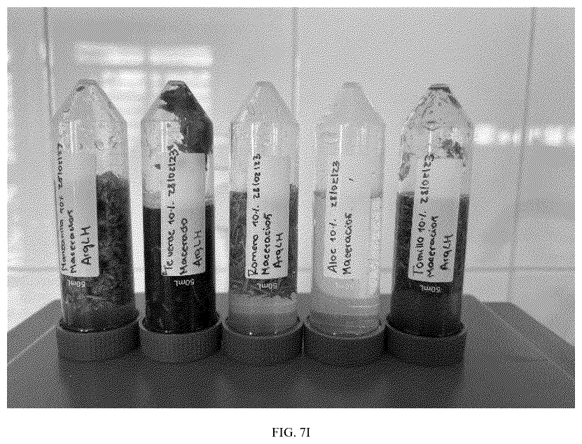

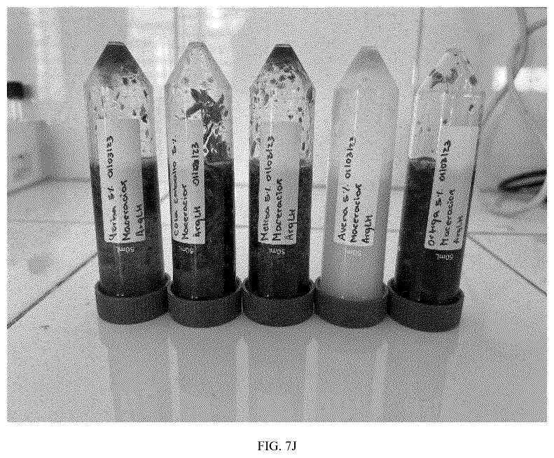

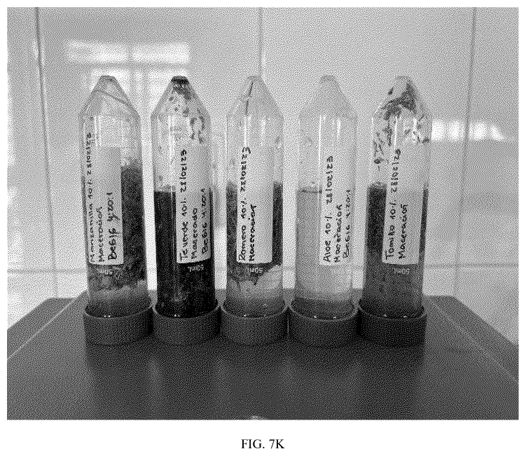

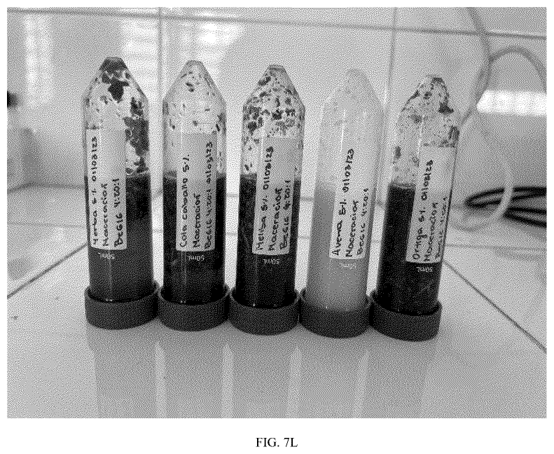

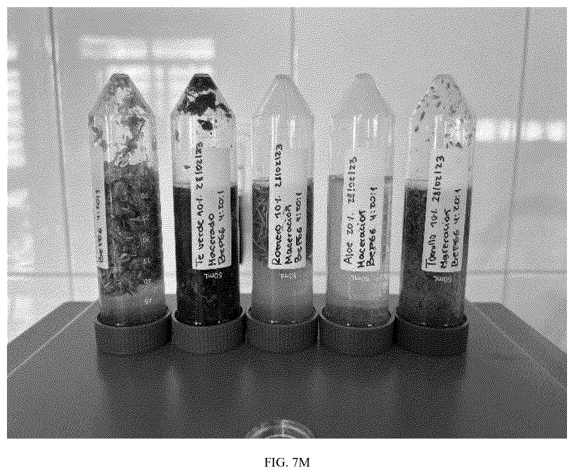

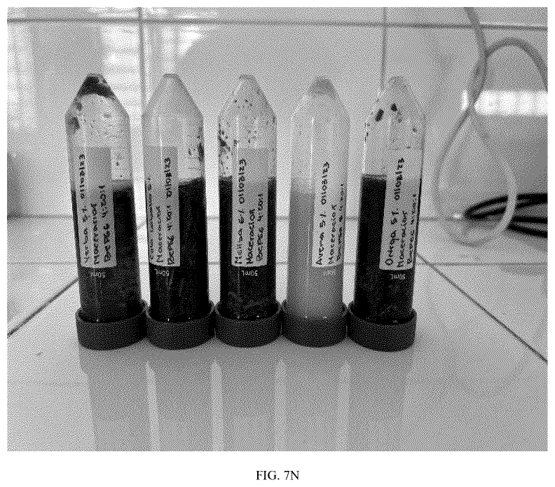

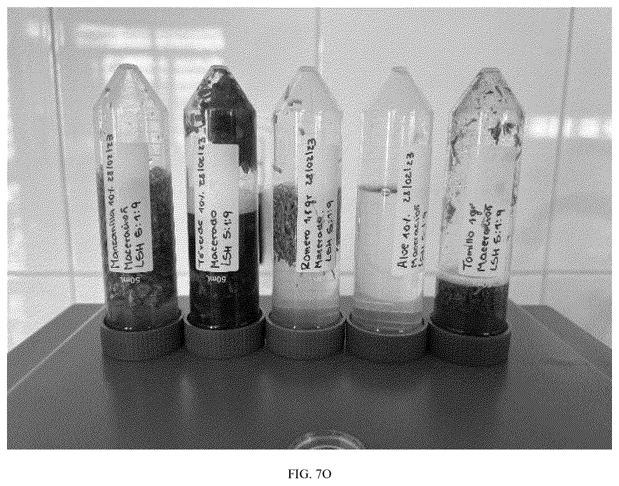

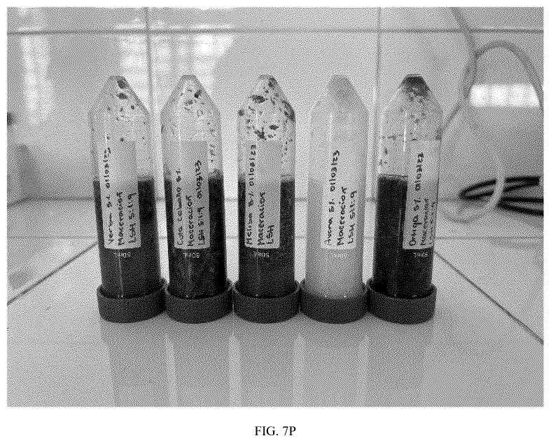

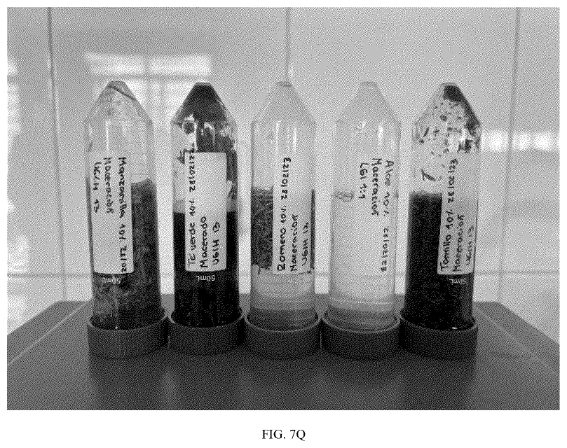

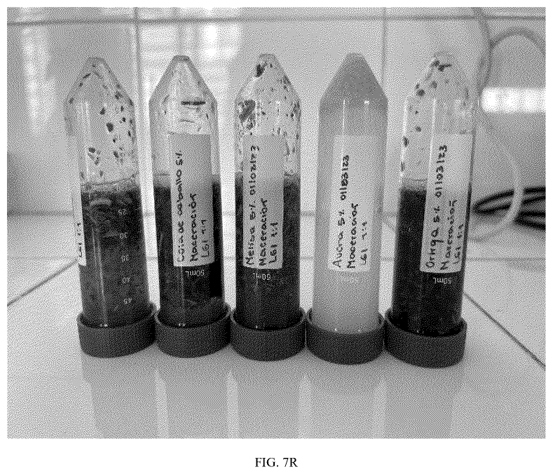

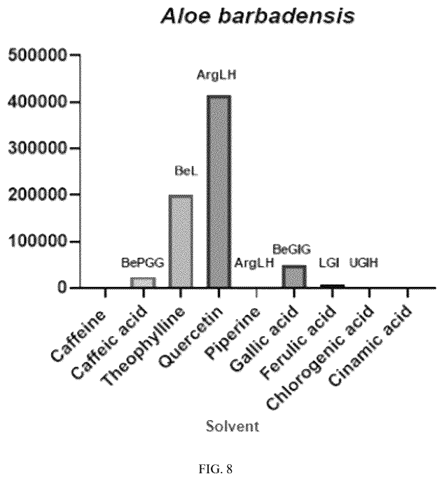

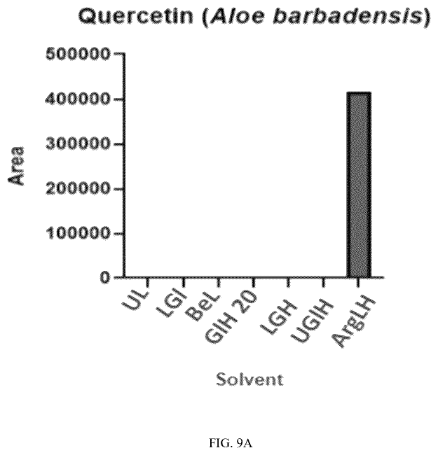

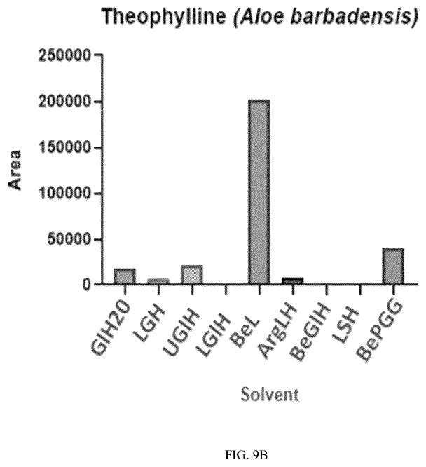

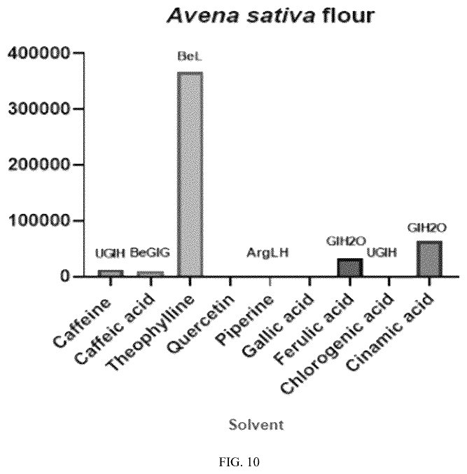

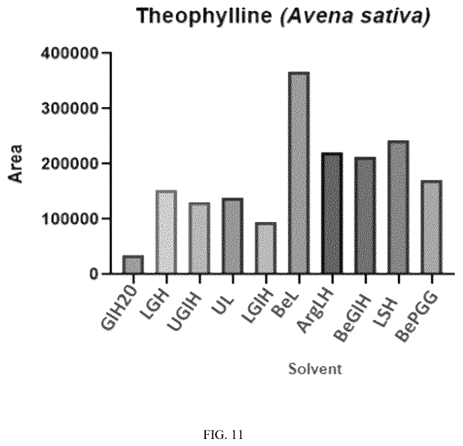

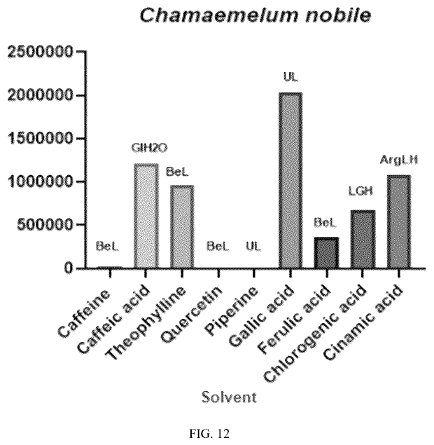

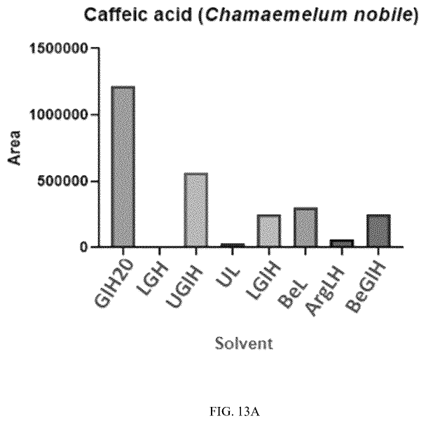

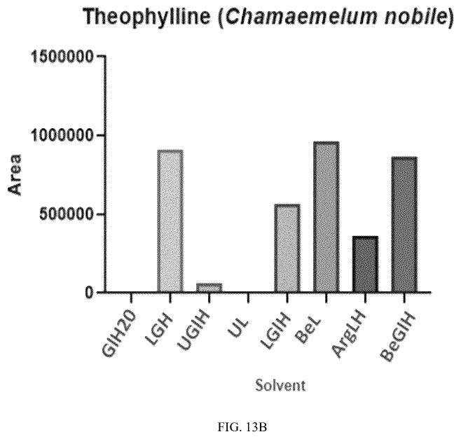

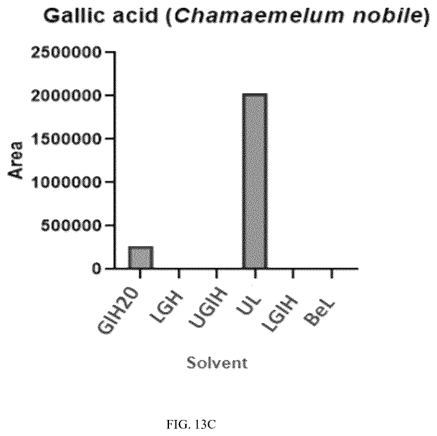

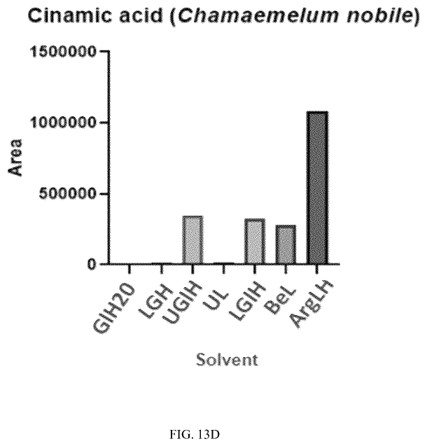

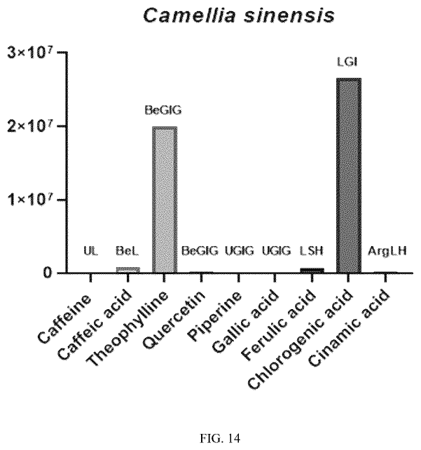

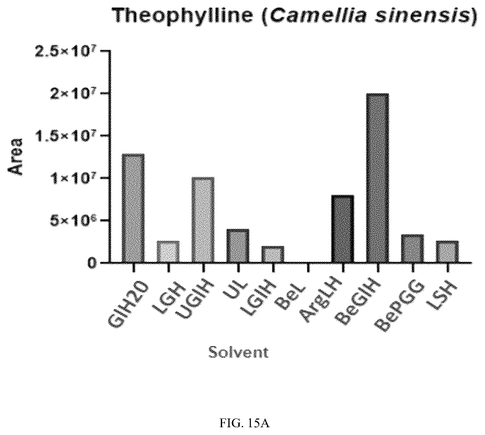

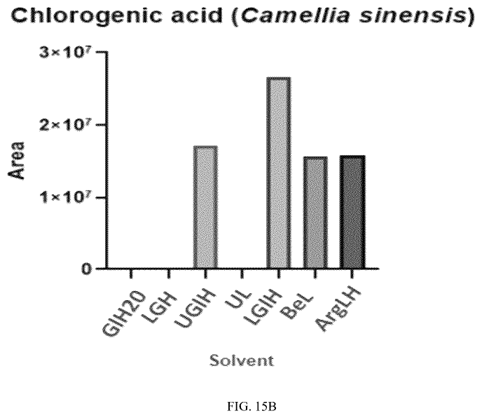

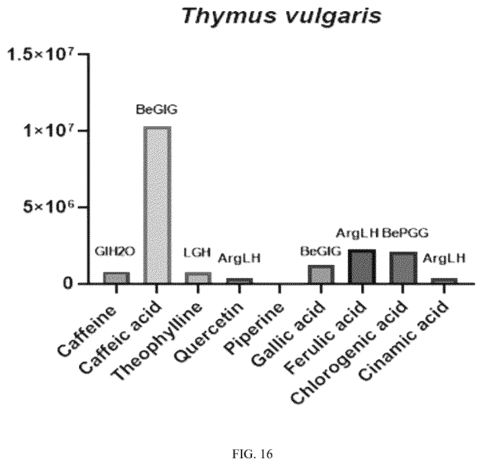

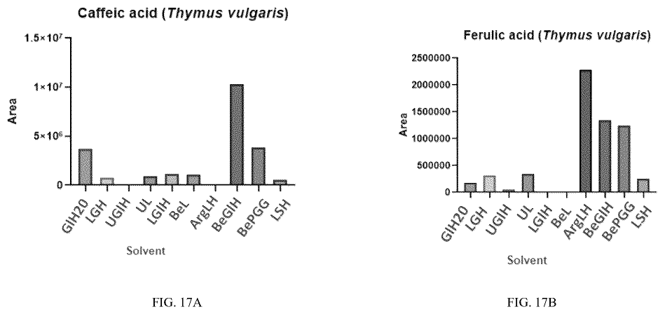

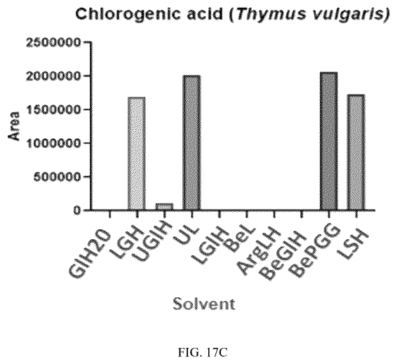

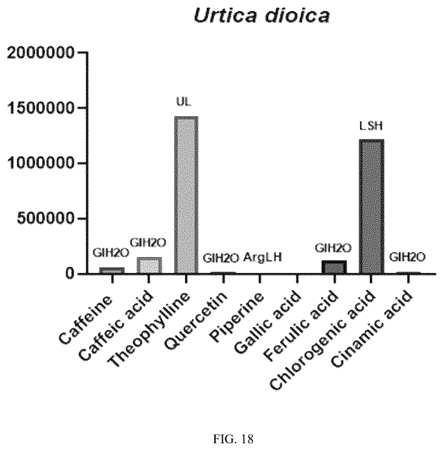

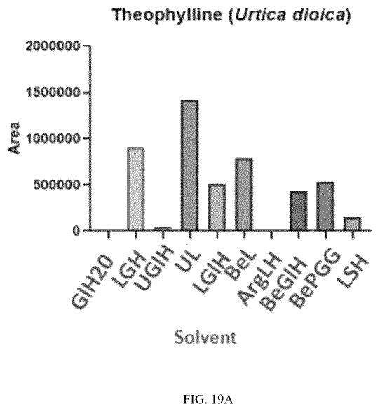

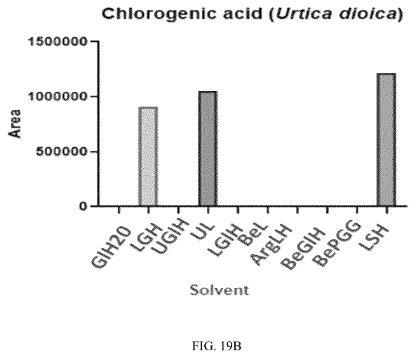

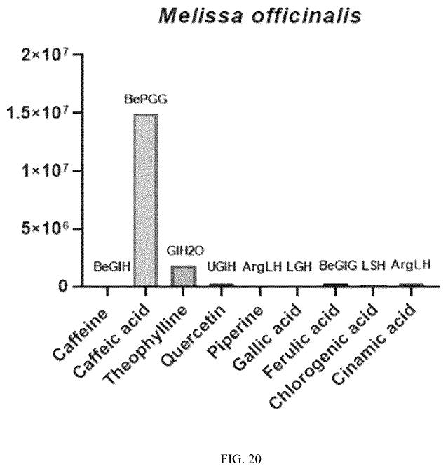

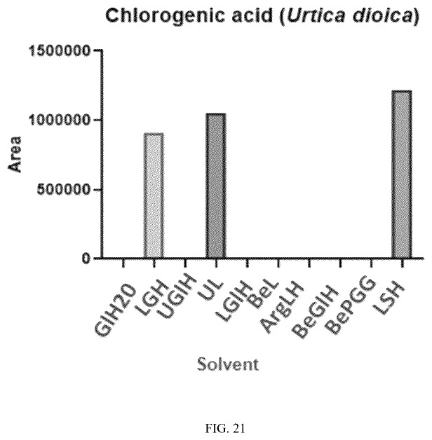

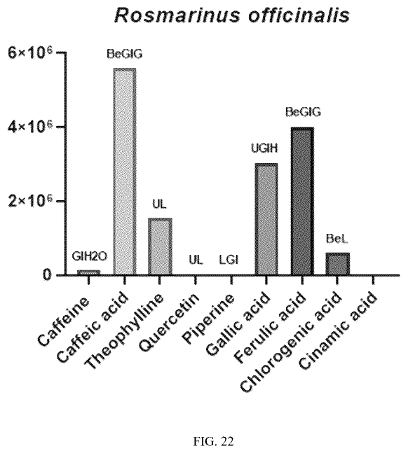

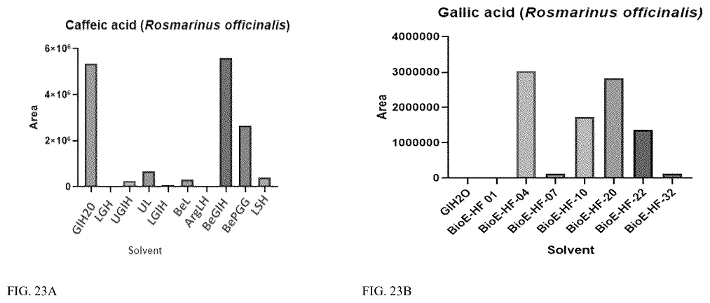

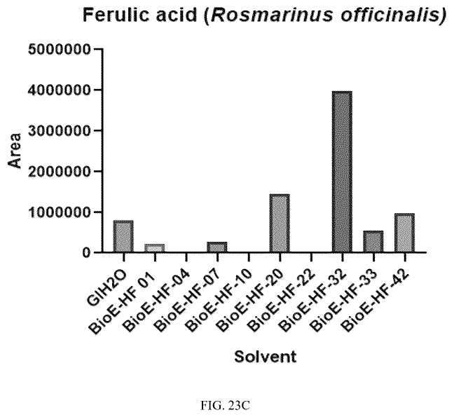

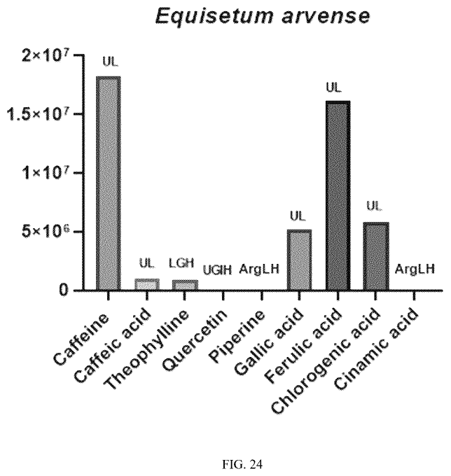

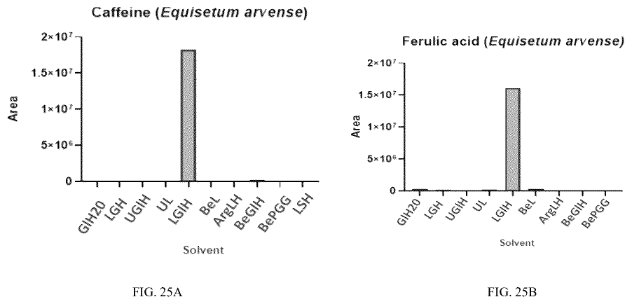

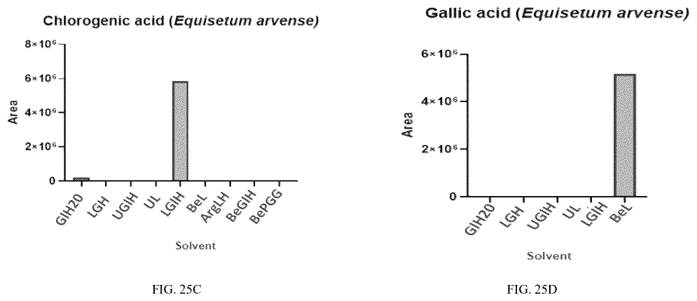

For the maceration extraction procedure, medicinal plants (2 g) and extracting solvent (20 g) were placed in a 50 mL centrifuge tube (plant/solvent ratio, 0.1 g/g). Immediately, the suspensions were vortexed for 30 s and then protected from light for 30 days at room temperature. The final concentrations were 5% w/w.
To compare, a positive control was analyzed (plant/solvent ratio, 0.1 g/g), and the extractive solvent used was glycerol:water (1:1 (v/v)).
The medicinal plants tested were:
Thymus vulgaris (Thyme) (see FIGS. 16 and 17A-C)
Rosmarinus officinalis (Rosemary) (see FIGS. 22 and 23A-C)
Matricaria chamomilla (Chamomile) (see FIG. 8)
Avena sativa (Oatmeal) (see FIGS. 10 and 11)
Aloe barbadensis (Aloe vera) (see FIGS. 9A-B)
Equisetum arvense (Horsetail) (see FIGS. 24 and 25A-D)
Melissa officinalis (Lemon balm) (see FIGS. 20 and 21)
Urtica dioica (Nettle) (see FIGS. 18 and 19A-B)
Ilex paraguariensis (Yerba mate)
Camellia sinensis (Green tea) (see FIGS. 14 and 15A-B)
   Except for Aloe Vera, all raw materials were dried before use. The bio-extracts are shown in FIGS. 7A-7R.
   Matricaria chamomilla Extraction Profile (See FIG. 8):
   The main polyphenol components of Aloe vera extract were theophylline and quercetin. The bio-solvents that best extracted these constituents are shown in the bar graphs that are FIG. 8.
   Avena sativa Extraction Profile (See FIGS. 10-11):
   The main polyphenol component of Avena sativa extract was theophylline. The bio-solvents that best extracted this constituent are shown in FIGS. 10 and 11.
   Chamaemelum nobile Profile Extract:
   The main polyphenol components of Chamaemelum nobile extract were caffeic acid, theophylline, gallic acid and cinnamic acid. The NADES that best extracted these constituents are shown in FIGS. 12 and 13A-D.
   The main polyphenol components of Camellia sinensis extract were theophylline and chlorogenic acid. The NADES that best extracted these constituents are shown in FIGS. 14 and 15A-B.
   Thymus vulgaris Extract Profile:
   The main polyphenol components of Thymus vulgaris extract were caffeic acid, ferulic acid and chlorogenic acid. The NADES that best extracted these constituents are shown in FIGS. 16 and 17A-C.
   Urtica dioica Profile Extract:
   The main polyphenol components of Urtica dioica extract were caffeic theophylline and chlorogenic acid. The NADES that best extracted these constituents are shown in FIGS. 18 and 19A-B.
   Melissa officinalis Extract Profile:
   The main polyphenol component of Thymus vulgaris extract was caffeic acid. The NADES that best extracted this constituent are shown in FIGS. 20 and 21.
   Rosmarinus officinalis Profile Extract:
   The main polyphenol components of Rosmarinus officinalis extract were caffeic acid, gallic acid and ferulic acid. The NADES that best extracted these constituents are shown in FIGS. 22 and 23A-C.
   Equisetum arvense Extraction Profile:
   The main polyphenol components of Equisetum arvense extract were caffeine, ferulic acid, chlorogenic acid, and gallic acid. The NADES that best extracted these constituents are shown in FIGS. 24 and 25A-D.

### CONCLUSIONS

The main polyphenols content of Aloe vera were theophylline and quercetin, best extracted by ArgLH and BeL, respectively.
The main polyphenol content of Avena sativa was theophylline, best extracted by BeL.
The main polyphenols content of Chamaemelum nobile were caffeic acid, theophylline, gallic acid and cinnamic acid, best extracted by GlH₂O (glycerol water mix) for caffeic acid, LGH, BeL, BeGl and BeGlH for theophylline, UL for gallic acid and ArgLH for cinnamic acid.
The main polyphenols content for Camellia sinensis were theophylline and chlorogenic acid, best extracted by BeGl, BeGlH and LGl, respectively.
The main polyphenols content of Thymus vulgaris were gallic acid, ferulic acid and chlorogenic acid, best extracted by BGl and BGlH for gallic acid, ArgLH for ferulic acid and UL and BePgG for chlorogenic acid.
The main polyphenols content of Urtica dioica were theophylline and chlorogenic acid, best extracted by UL and LSH, respectively. It could be observed that a correlation existed between the sample matrix and the extraction because a very good extraction of both polyphenols could be attained with LGH and UL.
The main polyphenol content of Melissa officinalis was caffeic acid, best extracted by BePgG.
The main polyphenols content of Rosmarinus officinalis were caffeic acid, gallic acid and ferulic acid, best extracted by BeGl, UGlH and BeGlH, respectively.
The main polyphenols content of Equisetum arvense were caffeine, ferulic acid, chlorogenic acid and gallic acid, all best extracted by LGl. There was also found to be a correlation between the matrix and the polyphenolic extraction.
For the majority of the polyphenols assayed, the best results were obtained with NADES improving (several times) the extraction with hydro-glyceric mixture.

In an embodiment, the present invention relates to being able to extract natural products from plants and/or plant products using NADES compositions. In many instances, the use of NADES for these extractions provides a pathway that is not only less toxic, but may confer other advantageous properties, such as being safe, non-flammable, inexpensive, less harmful to the environment, and in many cases being biodegradable. The use of NADES compositions for extraction purposes in many instances provides results that are comparable to the more traditional (but also more caustic) methodologies, and in some instances may actually provide results that surpass the use of the more traditional methodologies. Not only does the use of NADES compositions provide a safe method, but the components used in the NADES compositions, which may be used as pH regulators, and they tend to be food grade, meaning that they have little to no negative effect on the finished product and can be consumed and/or used by the consumer.

In an embodiment, the present invention relates to a method of extracting one or more natural products from a plant or plant product, the method comprising adding a NADES to the plant or the plant product to extract the one or more natural products, and using one or more of heat or time to facilitate isolation of the one or more natural products.

In a variation, the plant product is lignocellulosic biomass from a corn source and the method further comprises turning it into pulp, the method comprising extracting the corn source with a NADES. In a variation, the ratio of the corn source to the NADES is 20% w/w. In a variation, the method further comprises using heat. In a variation, the lignocellulosic biomass is heated to a temperature of 90° C. to 120° C. In a variation, the plant or plant product being extracted is heated to a temperature of at least 60° C. In a variation, the method comprises an extraction time of 2-4 hours. In a variation, the NADES comprises one or more of a mix of a) choline chloride and lactic acid b) betaine and lactic acid c) glycerol, lactic acid, and oxalic acid.

In an embodiment, the corn source is corn husk. In a variation, the plant or plant product derives from corn, thyme, rosemary, chamomile, oatmeal, aloe vera, horsetail, lemon balm, nettle, yerba mate, or green tea. In a variation, one or more natural products is a protein, a carbohydrate, an oil, or a polyphenol. In a variation, the one or more natural products is one or more of caffeic acid, caffeine chlorogenic acid, cinnamic acid, ferulic acid, gallic acid, pectin, quercetin, and/or theophylline. In a variation, the NADES is one or more of:
LGH: lactic acid, dextrose, and water
UGlH: urea, glycerol, and water
UL: urea and lactic acid
LGl: lactic acid and glycerol
BeL: betaine and lactic acid
ArgCH: L-arginine, citric acid, and water
ArgLH: L-arginine, lactic acid, and water
BeGlG: betaine, glycerol, and dextrose
BeGl: betaine, glycerol
BePGH: betaine, polyethylene glycol and water
LSH: lactic acid, sorbitol, and water.
   In a variation, the molar ratio of the NADES is as shown in parentheses below:
LGH: lactic acid, dextrose, and water (molar ratio: 5:1:9)
UGlH: urea, glycerol, and water (molar ratio: 1:1.2:1.2)
UL: urea and lactic acid (molar ratio: 1:2)
LGl: lactic acid and glycerol (molar ratio: 1:1)
BeL: betaine and lactic acid (molar ratio: 1:1)
ArgCH: L-arginine, citric acid, and water (molar ratio: 1:1:6.45)
ArgLH: L-arginine, lactic acid, and water (molar ratio: 1:1:9)
BeGlG: betaine, glycerol, and dextrose (molar ratio: 4:20:1)
   BeGl: betaine, glycerol (molar relation: 1:4)
BePGH: betaine, polyethylene glycol and water (molar ratio: 4:20:1)
LSH: lactic acid, sorbitol, and water (molar ratio: 5:1:9).

In a variation, the method comprises heating the plant or plant product and the NADES to a temperature of at least 60° C., and extracting the one or more natural products for a time period of at least 30 minutes.

In a variation, the NADES used when the temperature is at least 60° C., and the time period is at least 30 minutes, is one or more of:
lactic acid, dextrose, and water
urea, glycerol, and water
urea and lactic acid
lactic acid and glycerol
betaine and lactic acid
L-arginine, citric acid, and water
L-arginine, lactic acid, and water
betaine, glycerol, and dextrose
betaine, polyethylene glycol and water
lactic acid, sorbitol, and water
choline chloride and lactic acid
betaine and lactic acid, and/or
glycerol, lactic acid, and oxalic acid.

In a variation, the one or more natural products is one or more of caffeic acid, caffeine chlorogenic acid, cinnamic acid, ferulic acid, gallic acid, pectin, quercetin, and/or theophylline. In a variation, the temperature is at least 90° C., and the time period is at least one hour.

In a variation, the oil is derived from grapeseed.
In an embodiment, the present invention relates to a NADES composition that is:
lactic acid, dextrose, and water
urea, glycerol, and water
urea and lactic acid
lactic acid and glycerol
betaine and lactic acid
L-arginine, citric acid, and water
L-arginine, lactic acid, and water
betaine, glycerol, and dextrose
betaine, polyethylene glycol and water
lactic acid, sorbitol, and water
choline chloride and lactic acid
betaine and lactic acid, or
glycerol, lactic acid, and oxalic acid.

It should be understood and it is contemplated and within the scope of the present invention that any feature that is enumerated above can be combined with any other feature that is enumerated above as long as those features are not incompatible. Whenever ranges are mentioned, any real number that fits within the range of that range is contemplated as an endpoint to generate subranges. In any event, the invention is defined by the below claims.

We claim:

1. A method of extracting one or more natural products from a plant or plant product, the method comprising adding a NADES to the plant or the plant product to extract the one or more natural products, and using one or more of heat or time to facilitate isolation of the one or more natural products.

2. The method of claim 1, wherein the plant product is lignocellulosic biomass from a corn source and the method further comprises turning it into pulp, the method comprising extracting the corn source with a NADES.

3. The method of claim 2, wherein a ratio of the corn source to the NADES is 20% w/w.

4. The method of f claim 2, wherein the method further comprises using heat.

5. The method of claim 4, wherein the lignocellulosic biomass is heated to a temperature of 90° C. to 120° C.

6. The method of claim 1, wherein the plant or plant product being extracted is heated to a temperature of at least 60° C.

7. The method of claim 2, wherein the method comprises an extraction time of 2-4 hours.

8. The method of claim 2, wherein the NADES comprises one or more of a mix of a) choline chloride and lactic acid b) betaine and lactic acid c) glycerol, lactic acid, and oxalic acid.

9. The method of claim 2, wherein the corn source is corn husk.

10. The method of claim 1, wherein the plant or plant product derives from corn, thyme, rosemary, chamomile, oatmeal, aloe vera, horsetail, lemon balm, nettle, yerba mate, or green tea.

11. The method of claim 10, wherein the one or more natural products is a protein, a carbohydrate, an oil, or a polyphenol.

12. The method of 11, wherein the one or more natural products is one or more of caffeic acid, caffeine chlorogenic acid, cinnamic acid, ferulic acid, gallic acid, pectin, quercetin, and/or theophylline.

13. The method of claim 12, wherein the NADES is one or more of:
LGH: lactic acid, dextrose, and water
UGlH: urea, glycerol, and water
UL: urea and lactic acid
LGl: lactic acid and glycerol
BeL: betaine and lactic acid
ArgCH: L-arginine, citric acid, and water
ArgLH: L-arginine, lactic acid, and water
BeGlG: betaine, glycerol, and dextrose
BePGH: betaine, polyethylene glycol and water
LSH: lactic acid, sorbitol, and water.

14. The method of claim 13, wherein a molar ratio of the NADES is:
LGH: lactic acid, dextrose, and water (molar ratio: 5:1:9)
UGlH: urea, glycerol, and water (molar ratio: 1:1.2:1.2)
UL: urea and lactic acid (molar ratio: 1:2)
UGl: lactic acid and glycerol (molar ratio: 1:1)
BeL: betaine and lactic acid (molar ratio: 1:1)
ArgCH: L-arginine, citric acid, and water (molar ratio: 1:1:6.45)
ArgCH: L-arginine, lactic acid, and water (molar ratio: 1:1:9)
BeGlG: betaine, glycerol, and dextrose (molar ratio: 4:20:1)
BePGH: betaine, polyethylene glycol and water (molar ratio: 4:20:1)
LSH: lactic acid, sorbitol, and water (molar ratio: 5:1:9).

15. The method of claim 1, wherein the method comprises heating the plant or plant product and the NADES to a temperature of at least 60° C. and extracting one or more natural products for a time of at least 30 minutes.

16. The method of claim 15, wherein the NADES is one or more of:
lactic acid, dextrose, and water
urea, glycerol, and water
urea and lactic acid
lactic acid and glycerol
betaine and lactic acid
L-arginine, citric acid, and water
L-arginine, lactic acid, and water
betaine, glycerol, and dextrose
betaine, polyethylene glycol, and water
lactic acid, sorbitol, and water
choline chloride and lactic acid
betaine and lactic acid, and/or
glycerol, lactic acid, and oxalic acid.

17. The method of claim 16, wherein the one or more natural products is one or more of caffeic acid, caffeine chlorogenic acid, cinnamic acid, ferulic acid, gallic acid, pectin, quercetin, and/or theophylline.

18. The method of claim 15, wherein the temperature is at least 90° C., and the time period is at least one hour.

19. The method of claim 11, wherein the oil is derived from grapeseed.

20. A NADES that consists of:
lactic acid, dextrose, and water
urea, glycerol, and water
urea and lactic acid
lactic acid and glycerol
betaine and lactic acid
L-arginine, citric acid, and water
L-arginine, lactic acid, and water
betaine, glycerol, and dextrose
betaine, polyethylene glycol and water
lactic acid, sorbitol, and water
choline chloride and lactic acid
betaine and lactic acid, or
glycerol, lactic acid, and oxalic acid.
